(19) United States
(12) Patent Application Publication
(10) Pub. No.: US 2025/0108116 A1
(43) Pub. Date: Apr. 3, 2025

(54) GLYCOSYL BARBITURATE-BASED GELLING AGENTS

(71) Applicant: CENTRE NATIONAL DE LA RECHERCHE SCIENTIFIQUE, Paris (FR)

(72) Inventors: Sami HALILA, Beaucroissant (FR); Robin BRAHMI, Saint-Martin-d'Hères (FR)

(21) Appl. No.: 18/730,421

(22) PCT Filed: Jan. 20, 2023
(86) PCT No.: PCT/EP2023/051377

§ 371 Date: Jul. 19, 2024

(30) Foreign Application Priority Data
Jan. 21, 2022 (FR) 2200507

(51) Int. Cl. A61K 47/26; A61K 9/00; A61K 9/06

(52) U.S. Cl. CPC A61K 47/26; A61K 9/0014; A61K 9/06

(57) ABSTRACT

The use, as gelling agent, of a compound having the general formula (I): in which: —Sacc represents a monosaccharide or polysaccharide comprising up to 20 sugar units, in pyranose and/or furanose form and of the L and/or D series, said monosaccharide or polysaccharide having at least one free hydroxyl function; —X₁⁺ represents a cation chosen from the group consisting of: Na⁺, Li⁺, K⁺, ⁺NR³R⁴R⁵R⁶, and R⁷—NH₃, R³, R⁴, R⁵ and R⁶, which may be identical or different, representing a (C₁-C₂₀)alkyl group, R⁷ representing a (C₁-C₂₀)alkyl group, optionally including at least one unsaturation, said alkyl group being optionally substituted with at least one (C₆-C₁₀)aryl group, —R² represents in particular H or methyl, and also the tautomeric forms thereof.

The object of the present invention is novel gelling agents based on glycosyl barbiturates, known by the scientific name β-C-glycoside pyrimidinetrione, as well as a method for preparing an organogel or oleogel by the use of said gelling agents based on glycosyl barbiturates.

Organogelling agents are small organic molecules capable of texturing and as far as gelling all kinds of organic solvents or oily phases, even at relatively low mass concentrations (less than 1% by mass).

At the present time numerous molecules exist that can be used as organogelling agents but these have a certain number of drawbacks.

This is because synthesizing these organogelling agents requires multiple steps, as well as the use of toxic solvents and/or reagents. Moreover, the majority of these organogelling agents have a hydrophobic nature, which makes them insoluble in an aqueous medium and therefore not “washable”. For many organogelling agents, their chemical and enzymatic stability is a brake on the development thereof.

Moreover, organogelling agents of a polymeric nature such as ethylcellulose furthermore have the drawback of leaving a film after they are applied.

Finally, an organogelling agent is often specific to an application and organogelling agents have only the single function of texturing or gelling.

There is therefore at the present time a need for an organogelling agent not having the aforementioned drawbacks, and therefore in particular providing an organogelling compound with a biosourced nature, that can easily be synthesized in an ecoresponsible manner and being chemically and enzymatically stable.

The aim of the present invention is therefore to provide an organogelling agent that can be synthesized in a single step and under environmentally friendly conditions, in particular in the presence of aqueous solvents, without the formation of by-products, is easy to purify and to obtain a quantitative yield.

Another aim of the present invention is to provide an organogelling agent that is perfectly soluble in water and therefore washable at the end of application for dermal treatments, for example.

Another aim of the present invention is to provide an organogelling agent that is chemically and enzymatically stable, and optionally with an easily modulatable gelling ability.

Thus the present invention relates to the use, as a gelling or texturing agent, of a compound having the following general formula (I):

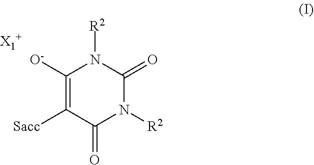

wherein:
Sacc represents a monosaccharide or polysaccharide including up to 20 sugar units, in pyranose and/or furanose form and of the L and/or D series, said mono- or polysaccharide having at least one free hydroxyl function;
X₁⁺ represents a cation selected from the group consisting of: Na⁺, Li⁺, K⁺, ⁺NR³R⁴R⁵R⁶, R⁷—NH₃⁺, ⁺PR³R⁴R⁵R⁶ and ⁺SR³R⁴R⁵,
R³, R⁴, R⁵ and R⁶, identical or different, represent a (C₁-C₂₀)alkyl group, optionally including at least one unsaturation, said alkyl group optionally being substituted with at least one (C₆-C₁₀)aryl group, in particular a phenyl group,
R⁷ representing a (C₁-C₂₀)alkyl group, optionally including at least one unsaturation, said alkyl group optionally being substituted with at least one (C₆-C₁₀)aryl group, in particular a phenyl group,
R² represents H, methyl, or a (C₁-C₂₀)alkyl group optionally including at least one unsaturation, said alkyl group optionally being substituted with at least one (C₆-C₁₀)aryl group, in particular a phenyl group,
and also the tautomeric forms thereof.

Thus the compounds used as texturing or gelling agents according to the invention (or organogelling agents) are C-glycoside derivatives that are chemically and enzymatically stable by virtue of the non-natural C-glycoside bond.

The gelling capability of these compounds can easily be modulated by acting on the concentration thereof and the nature of the positive counter-ion, for example by simple mixing.

The organogelling agents according to the invention can be used for example in cosmetic compositions and perfumes or pharmaceutical or dermo-pharmaceutical compositions, but also in the food field or in the lubricant, paint or building industries or for maintaining painting canvases.

Texture agents are additives present in cosmetic, food or other formulae in order to increase the consistency, the viscosity and the sensoriality of the finished products. Texturing agents are in particular synthesis polymers such as polyacrylates, biosourced polymers such as polysaccharides and derivatives thereof, gums and lipids derived from oils or butters, esters or waxes.

A gelling agent may be texturing and this will depend on the concentration thereof in the medium. Below its minimum gelling concentration for a given fatty phase, it will be texturing, and above or equal it will be gelling.

The tautomeric form of the general formula (I) is as follows:

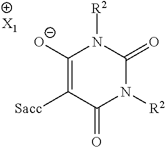

According to the invention, (Cₜ-C_{z}) group means a group comprising a carbon chain that can have from t to z carbon atoms, for example C₁-C₆, a carbon chain that can have from 1 to 6 carbon atoms.

According to the invention, the term “alkyl” designates saturated hydrocarbon aliphatic groups, linear or branched, comprising, unless mentioned to the contrary, from 1 to 20 carbon atoms. By way of examples, mention can be made of the methyl, ethyl, n-propyl, isopropyl, butyl, isobutyl, tert-butyl or pentyl groups.

According to one embodiment, the alkyl groups according to the invention can be interrupted by at least one unsaturation.

The alkyl groups of the invention can also be substituted with at least one aryl group.

According to the invention, the aryl groups are cyclic aromatic groups comprising between 6 and 10 carbon atoms. By way of examples of aryl groups, mention can be made of the phenyl or naphthyl groups.

Preferably, in formula (I), R³, R⁴, R⁵ and R⁶, identical or different, represent a (C₁-C₂₀)alkyl group, or a benzyl group.

Preferably, in formula (I), R⁷ represents a (C₁-C₂₀)alkyl group.

Preferably, in formula (I), R² represents H or methyl, preferably methyl.

Preferably, in formula (I), X₁⁺ represents a cation selected from the group consisting of: Na⁺, Li⁺ and K⁺, and is preferentially Na⁺.

In one embodiment, the cation X₁⁺ can be selected from polycharged derivatives such as for example polyammonium ions.

In one embodiment, in formula (I), Sacc is selected from the group consisting of D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine, N-acetyl-D-galactosamine, D-maltose, D-lactose, D-cellobiose, D-maltotriose, D-iduronic acid, D-glucuronic acid with a hexosamine selected from D-galactosamine, D-glucosamine, N-acetyl-D-galactosamine or N-acetyl-D-glucosamine, xylobiose and oligomers thereof (from xylotriose to xylohexaose).

Preferably, in formula (I), Sacc is D-glucose.

The C-glycoside derivatives having the formula (I) according to the present invention can be prepared, for example, in accordance with the method described by Gonzales M. A. et al. Carbohydrate Research 1986, 158, pp 58-66 and by Wulff G. et al. Carbohydrate Research 1994, 257, pp 81-95.

Among the C-glycoside derivatives having the formula (I) used according to the invention, the following compounds are particularly preferred:
sodium 1,3-dimethyl-2,6-dioxo-5-[(2S,3R,4R,5S,6R)-3,4,5-trihydroxy-6-(hydroxy-methyl)tetrahydro-2H-pyran-2-yl]-1,2,3,6-tetrahydropyrimidin-4-olate:

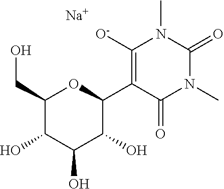 [SMILES: Cn1c([O-])c([C@@H]2O[C@H](CO)[C@@H](O)[C@H](O)[C@H]2O)c(=O)n(C)c1=O ; [Na+]]

sodium 2,6-dioxo-5-[(2S,3R,4R,5S,6R)-3,4,5-trihydroxy-6-(hydroxymethyl)tetrahydro-2H-pyran-2-yl]-1,2,3,6-tetrahydropyrimidin-4-olate

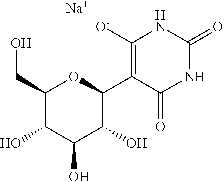 [SMILES: [Na+] ; O=c1[nH]c([O-])c([C@@H]2O[C@H](CO)[C@@H](O)[C@H](O)[C@H]2O)c(=O)[nH]1]

sodium 1,3-dimethyl-2,6-dioxo-5-[(2S,3R,4R,5R,6R)-3,4,5-trihydroxy-6-(hydroxymethyl)tetrahydro-2H-pyran-2-yl]-1,2,3,6-tetrahydropyrimidin-4-olate:

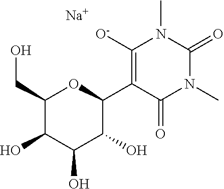 [SMILES: Cn1c([O-])c([C@@H]2O[C@H](CO)[C@H](O)[C@H](O)[C@H]2O)c(=O)n(C)c1=O ; [Na+]]

sodium 1,3-dimethyl-2,6-dioxo-5[(2S,3R,4S,5R)-3,4,5-trihydroxytetrahydro-2H-pyran-2-yl]-1,2,3,6-tetrahydropyrimidin-4-olate:

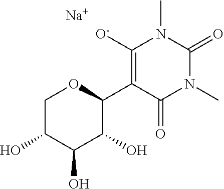 [SMILES: Cn1c([O-])c([C@@H]2OC[C@@H](O)[C@H](O)[C@H]2O)c(=O)n(C)c1=O ; [Na+]]

sodium 2,6-dioxo-5-[(2S,3R,4S,5R)-3,4,5-trihydroxytetrahydro-2H-pyran-2-yl]-1,2,3,6-tetrahydropyrimidin-4-olate

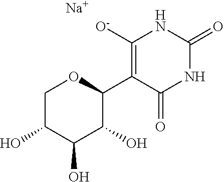 [SMILES: [Na+] ; O=c1[nH]c([O-])c([C@@H]2OC[C@@H](O)[C@H](O)[C@H]2O)c(=O)[nH]1]

sodium 1,3-dimethyl-2,6-dioxo-5-[(2S,3R,4S,5R,6R)-3,4,5-trihydroxy-6-methyl-tetrahydro-2H-pyran-2-yl]-1,2,3,6-tetrahydropyrimidin-4-olate:

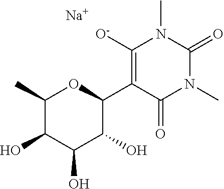 [SMILES: C[C@H]1O[C@@H](c2c([O-])n(C)c(=O)n(C)c2=O)[C@H](O)[C@@H](O)[C@H]1O ; [Na+]]

Preferably, the compound having the formula (I) according to the invention complies with the following formulae:

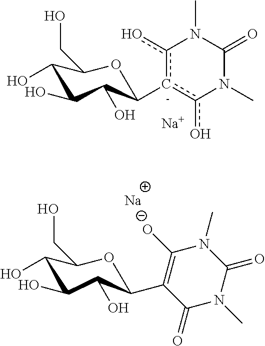

Several advantages are associated with the use of this class of molecule that belongs to the C-glycoside family.

The aforementioned preferred glycosyl barbiturate is for example obtained in a single synthesis step in water and characterised by a glucose unit bonded by a C-glycoside bond to N,N-dimethylbarbituric acid. Purification thereof is obtained by simple precipitation of the reaction medium with a quantitative yield, making producing same easy and practical from the industrial point of view.

The starting reagents for preparing this molecule are available commercially.

The compounds of the invention (or glyco-gelling agents) texture as far as gelling both organic solvents with different polarities and oils and the gelling capability thereof can easily be modulated by the nature of the associated cationic counter-ion. Gelling can be induced by ultrasound, simple mixing or by a heating cycle followed by cooling to ambient temperature, as explained below.

According to one embodiment, the compound having the formula (I) is used as a gelling agent of an organic liquid or mixture of liquids, said organic liquid being selected from organic solvents and oils.

According to the invention, an organic liquid is a liquid with a carbon chain or including carbon. As indicated above, it may be an organic solvent, an oil or a mixture thereof.

According to one embodiment, the organic liquid is an organic solvent selected from the group consisting of alcohols, ketones, aliphatic, aromatic or halogenated hydrocarbons, esters, nitrogen derivatives, ethers, silicones, aliphatic carboxylic acids and the ester derivatives thereof, amides, lipids or glycerides. Preferably, the organic solvent is selected from ethanol, propanol, glycerol, butanol, octanol, isopropanol, anisole, octyldodecanol, ethylacetate, acetone, tetrahydrofuran, oleic acid, linoleic acid, palmitic acid and esters of adipate, stearate or caprate.

According to one embodiment, the organic liquid is an oil selected from vegetable or animal oils, and natural or synthetic mineral oils. Preferably, the oil is selected from colza oil, castor oil, jojoba oil, essential oil of lavender, palm oil, olive oil, sunflower oil, paraffin or liquefied petroleum.

Preferably, the organic liquid is an organic solvent selected from ethanol, propanol, butanol, glycerol, octanol, isopropanol, anisole, ethylacetate and tetrahydrofuran, or an oil selected from colza oil or essential oil of lavender (EO of lavender).

The present invention also relates to the aforementioned use wherein the compound having the formula (I) is used as an agent for texturing or gelling a mixture of organic liquids, preferably comprising at least one oil and/or at least one organic solvent.

For example, by way of mixture, mention can be made of a mixture comprising essential oil of lavender and ethanol, or a mixture comprising colza oil, octanol and isopropanol, or a silicone oil and a fatty ester but also a mixture of two organic solvents such as a mixture of octanol and ethanol.

Preferably, in the context of the use as defined above, the proportion by weight of the compound having the formula (I) is between 0.1% and 10%, preferably 1% to 5%, by weight with respect to the weight of the organic liquid or mixture of organic liquids.

The present invention also relates to a method for preparing an organogel or oleogel comprising a step of putting a compound having the formula (I) in the presence of an organic liquid or mixture of organic liquids, said liquid being selected from organic solvents and oils, and being as defined above,
said compound having the formula (I) complying with the following formula:

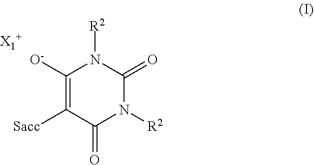

wherein:
Sacc represents a monosaccharide or polysaccharide including up to 20 sugar units, in pyranose and/or furanose form and of the L and/or D series, said mono- or polysaccharide having at least one free hydroxyl function;
X₁⁺ represents a cation selected from the group consisting of: Na⁺, Li⁺, K⁺, ⁺NR³R⁴R⁵R⁶, R⁷—NH₃⁺, ⁺PR³R⁴R⁵R⁶ and ⁺SR³R⁴R⁵,
R³, R⁴, R⁵ and R⁶, identical or different, representing a (C₁-C₂₀)alkyl group, optionally including at least one unsaturation, said alkyl group optionally being substituted with at least one (C₆-C₁₀)aryl group, in particular a phenyl group,
R⁷ representing a (C₁-C₂₀)alkyl group, optionally including at least one unsaturation, said alkyl group optionally being substituted with at least one (C₆-C₁₀)aryl group, in particular a phenyl group,
R² represents H, methyl, or a (C₁-C₂₀)alkyl group optionally including at least one unsaturation, said alkyl group optionally being substituted with at least one (C₆-C₁₀)aryl group, in particular a phenyl group,
and also the tautomeric forms thereof.

According to the invention, the term organogel designates a gel of an organic solvent and the term oleogel designates a gelled oil.

According to one embodiment, the method of the invention comprises adding an aqueous solution of the compound having the formula (I) in the organic liquid or mixture of organic liquids.

Preferably, the addition step furthermore comprises an ultrasound treatment step or a step of heating or cooling the organic liquid or mixture of organic liquids.

According to one embodiment of the method according to the invention, the organic liquid or mixture of organic liquids comprises an additional compound selected from dyes or pigments, an opacifier, an organic or mineral UV filter, odorising molecules and pharmaceutical active principles, such as Nile red, carmine red, metal oxides, ethylhexyl triazone, eucalyptol or doxorubicine.

### EXAMPLES

### Example 1: Determining the Correct Dosage for Forming 2% Gels (w/v)

10 mg of glucosyl barbiturate or sodium β-C-glucoside pyrimidinetrione are weighed in a flask of approximately 1.5 ml with a screw stopper. 450 μL of the selected solvent are added and then the mixture is sonicated for a few seconds in order to disperse the powder well in the solvent (in the majority of cases the glucosyl barbiturate is not soluble in the solvent). Next water is added by portions of 2 μL and between each addition the mixture is sonicated and lightly stirred by hand to see if the gel is forming. The sonication time varies according to the nature of the solvent. Once the gel has formed, it is possible for it not to be very homogeneous. In this case, it is necessary to be able to judge actions to be taken to obtain the gel. Often it is necessary: i) either to add a very small amount of water, heat and leave to cool, or ii) to heat and leave to cool while sonicating (if the glucosyl barbiturate precipitates while cooling). The mixture is next completed with the solvent and gelled to 500 μL.

### Example 2: Forming a Gel by Adding a Solvent+Water Mixture on the Glycobarbiturate Powder

10 mg of sodium glucosyl barbiturate is weighed, then the mixture of solvent and water is added in the correct proportions defined above in Example 1. The mixture is next either sonicated cold in an ultrasound bath, or heated and leaved to cool to obtain the gel, or are briskly stirred at ambient temperature and then left to rest.

For example, it is necessary to add 40 μL of water in 500 μL of ethanol, 60 μL of water in 440 μL of isopropanol, 45 μL of water in 455 μL of butanol, 60 μL of water in 420 μL of acetone or 35 μL of water in 480 μL of propan-1-ol.

### Example 3: Forming a Gel by Adding a Concentrated Solution of Sodium Glucosyl Barbiturate to the Solvent

The solvent is placed in a receptacle under stirring and then the aqueous solution of sodium glucosyl barbiturate (1.47 M) is added in the proportions defined above in Example 1. The mixture is left to stir for a few seconds and then the stirring is stopped to allow the gel to form.

In this example, 80 μL of the aqueous solution of sodium glucosylbarbiturate (1.47 M) was added to 1 ml of 96% ethanol.

### Example 4: Forming a Gel with Oils or Apolar Solvents (Examples: Colza Oil, Toluene, Anisole, Etc.)

With solvents that are not miscible with water, it is necessary to add either another solvent that makes it possible to introduce the small amount of water necessary for forming gels (an example of a mixture that can accept a small amount of water without forming two phases: EO of lavender/ethanol), i.e. changing the nature of the counter-ion that is at the origin of the sodium by an ammonium alkyl salt. Three ammonium salts were tested, namely cetyltrimethylammonium bromide (CTAB), tetrabutylammonium bromide (TBAB) or Tequat® LC 90i (tallow esterquat). To find the conditions favourable for gelling, it is possible to use the same method as that explained in Example 1. Glucosyl barbiturate and an ammonium salt equivalent are weighed before adding the solvent and adjusting the correct quantity of water.

The various results are described hereinafter in Table 1 below.

For example, 10 mg of sodium glucosyl barbiturate was mixed with either 9.4 mg of TBAB or 11 mg of CTAB. Next 400 μL of colza oil was added and then the whole was sonicated for dispersion, and finally 5 μL of water was added. Sonication makes it possible to form a gel instantaneously.

With on each occasion 10 mg of sodium glucosyl barbiturate weighed and 1 mol equivalent of ammonium salt, the protocol followed for the various solvent tests of Table 1 is the one detailed in Example 4.

TABLE 1

| Solvent 1 | Solvent 2 | Solvent 3 | H₂O | CTAB | TBAB | Tequat |
|---|---|---|---|---|---|---|
| μL | μL | μL | μL | mg | mg | 90 LCi |
| Ethanol | — | — | 40 | — | — | — |
| 500 | | | | | | |
| Isopropanol | — | — | 60 | — | — | — |
| 440 | | | | | | |
| 1-butanol | — | — | 45 | — | — | — |
| 155 | | | | | | |
| Acetone | — | — | 60 | — | — | — |
| 440 | | | | | | |
| 1-propanol | — | — | 35 | — | — | — |
| 480 | | | | | | |
| EO of lavender | Ethanol | — | 20 | — | — | — |
| 250 | 250 | | | | | |
| 1-octanol | Ethanol | — | 20 | — | — | — |
| 300 | 100 | | | | | |
| Colza oil | 1-octanol | Isopropanol | 10 | — | — | — |
| 250 | 50 | 230 | | | | |
| 1-octanol | — | — | 10 | — | 9.4 | — |
| 400 | | | | | | |
| Colza oil | — | — | 5 | — | 9.4 | — |
| 400 | | | | | | |
| Colza oil | Nile red | — | 5 | — | 9.4 | — |
| 490 | (THF) | | | | | |
| | 10 | | | | | |
| Anisole | — | — | 2 | — | 9.4 | — |
| 500 | | | | | | |
| Ethylacetate | Nile red | — | 20 | 11 | — | — |
| 490 | (THF) | | | | | |
| | 10 | | | | | |
| Colza oil | Nile red | — | 5 | 11 | — | — |
| 490 | (THF) | | | | | |
| | 10 | | | | | |
| THF | — | — | 8 | 11 | — | — |
| 500 | | | | | | |
| Octyldodecanol | — | — | 2 | — | 9.4 | — |
| 500 | | | | | | |
| Butylene glycol | — | — | 4 | — | 9.4 | — |
| 500 | | | | | | |
| Glycerol | — | — | 20 | 11 | — | — |
| 500 | | | | | | |
| Isododecane | — | — | 5 | 11 | — | — |
| 500 | | | | | | |
| Dimethicone | Ethylacetate | — | 10 | 11 | — | — |
| 250 | 250 | | | | | |
| Diisopropyl | — | — | 2 | 11 | 9.4 | — |
| adipate | | | | | | |
| 500 | | | | | | |
| Caprylic/capric | — | — | 2 | — | 9.4 | — |
| triglycerides | | | | | | |
| 500 | | | | | | |
| Coconut | — | — | 2 | — | — | 30 |
| alkanes | | | | | | |
| 500 | | | | | | |
| Dicaprylyl | — | — | — | — | — | 30 |
| carbonate | | | | | | |
| 500 | | | | | | |
| Jojoba oil | — | — | 2 | 11 | — | — |
| 500 | | | | | | |
| Castor oil | — | — | 2 | 11 | — | — |

What is claimed is:

1. An agent for texturing or gelling an organic liquid or mixture of organic liquids comprising a compound having the following general formula (I): 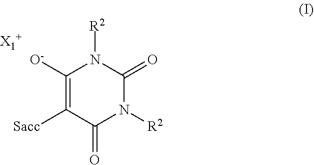
wherein:
Sacc represents a monosaccharide or polysaccharide including up to 20 sugar units, in pyranose and/or furanose form and of the L and/or D series, said mono- or polysaccharide having at least one free hydroxyl function;
X₁⁺ represents a cation selected from the group consisting of: Na⁺, Li⁺, K⁺, ⁺NR³R⁴R⁵R⁶, R⁷—NH₃⁺, ⁺PR³R⁴R⁵R⁶ and ⁺SR³R⁴R⁵,
R³, R⁴, R⁵ and R⁶, identical or different, represent a (C₁-C₂₀)alkyl group, optionally including at least one unsaturation, said alkyl group optionally being substituted with at least one (C₆-C₁₀)aryl group,
R⁷ representing a (C₁-C₂₀)alkyl group, optionally including at least one unsaturation, said alkyl group optionally being substituted with at least one (C₆-C₁₀)aryl group,
R² represents H, or a (C₁-C₂₀)alkyl group optionally including at least one unsaturation, said alkyl group optionally being substituted with at least one (C₆-C₁₀)aryl group,
and also the tautomeric forms thereof.

2. The agent according to claim 1, wherein, in formula (I), Sacc is selected from the group consisting of D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine, N-acetyl-D-galactosamine, D-maltose, D-lactose, D-cellobiose, D-maltotriose, D-iduronic acid, D-glucuronic acid with a hexosamine selected from D-galactosamine, D-glucosamine, N-acetyl-D-galactosamine or N-acetyl-D-glucosamine, xylobiose and oligomers thereof.

3. A composition comprising an organic liquid and the agent according to claim 1, wherein the organic liquid is selected from organic solvents and oils.

4. The composition according to claim 3, wherein the organic liquid is an organic solvent selected from the group consisting of alcohols, ketones, aliphatic, aromatic or halogenated hydrocarbons, esters, nitrogen derivatives, ethers, silicones, aliphatic carboxylic acids and the ester derivatives thereof, amides, lipids or glycerides, and is preferably selected from ethanol, propanol, glycerol, butanol, octanol, isopropanol, anisole, ethylacetate, octyldodecanol, tetrahydrofuran, acetone, oleic acid, linoleic acid, palmitic acid and esters of adipate, stearate or caprate.

5. The composition according to claim 4, wherein the oil is selected from vegetable or animal oils, and natural or synthetic mineral oils, and is preferably colza oil, essential oil of lavender, palm oil, olive oil, sunflower oil, castor oil, jojoba oil, paraffin or liquefied petroleum.

6. The composition according to claim 3, wherein the proportion by weight of the compound of formula (I) is between 0.1% and 10%, preferably from 1% to 5%, by weight with respect to the weight of the organic liquid or mixture of organic liquids.

7. A method for preparing an organogel or oleogel comprising a step of putting a compound of formula (I) in the presence of an organic liquid or mixture of organic liquids, said organic liquid or mixture of organic liquids being selected from the group consisting of alcohols, ketones, aliphatic, aromatic or halogenated hydrocarbons, esters, nitrogen derivatives, ethers, silicones, aliphatic carboxylic acids and the ester derivatives thereof, amides, lipids or glycerides, and is preferably selected from ethanol, Propanol, glycerol, butanol, octanol, isopropanol, anisole, ethylacetate, octyldodecanol, tetrahydrofuran, acetone, oleic acid, linoleic acid, palmitic acid and esters of adipate, stearate, caprate, vegetable or animal oils, and natural or synthetic mineral oils, and is preferably colza oil, essential oil of lavender, palm oil, olive oil, sunflower oil, castor oil, ioioba oil, paraffin or liquefied petroleum and mixtures thereof,
said compound having the formula (I) complying with the following formula: 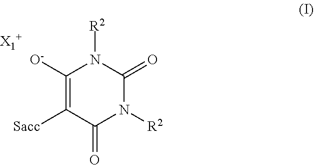
wherein:
Sacc represents a monosaccharide or polysaccharide including up to 20 sugar units, in pyranose and/or furanose form and of the L and/or D series, said mono- or polysaccharide having at least one free hydroxyl function;
X₁⁺ represents a cation selected from the group consisting of: Na⁺, Li⁺, K⁺, ⁺NR³R⁴R⁵R⁶, R⁷—NH₃⁺, ⁺PR³R⁴R⁵R⁶ and ⁺SR³R⁴R⁵,
R³, R⁴, R⁵ and R⁶, identical or different, represent a (C₁-C₂₀)alkyl group, optionally including at least one unsaturation, said alkyl group optionally being substituted with at least one (C₆-C₁₀)aryl group, in particular a phenyl group,
R⁷ representing a (C₁-C₂₀)alkyl group, optionally including at least one unsaturation, said alkyl group optionally being substituted with at least one (C₆-C₁₀)aryl group, in particular a phenyl group,
R² represents H, or a (C₁-C₂₀)alkyl group optionally including at least one unsaturation, said alkyl group optionally being substituted with at least one (C₆-C₁₀)aryl group, in particular a phenyl group,
and also the tautomeric forms thereof.

8. The method according to claim 7, further comprising adding an aqueous solution of the compound having the formula (I) in the organic liquid or mixture of organic liquids.

9. The method according to claim 8, wherein the adding step further comprises a step of treatment by ultrasound or a step of heating or cooling the organic liquid or mixture of organic liquids.

10. The method according to claim 7, wherein the organic liquid or mixture of organic liquids comprises an additional compound selected from dyes, odorising molecules and pharmaceutical active principles.
